(19) United States
(12) Patent Application Publication
(10) Pub. No.: US 2006/0275911 A1
(43) Pub. Date: Dec. 7, 2006

(54) Method and apparatus for moleclular analysis using nanostructure-enhanced Raman spectroscopy

(76) Inventors: Shih-Yuan Wang, Palo Alto, CA (US); Wei Wu, Mountain View, CA (US); Zhiyong Li, Palo Alto, CA (US)

Correspondence Address: HEWLETT PACKARD COMPANY, P O BOX 272400, 3404 E. HARMONY ROAD, INTELLECTUAL PROPERTY ADMINISTRATION, FORT COLLINS, CO, 80527-2400, US

(21) Appl. No.: 11/145,038

(22) Filed: Jun. 3, 2005

(51) Int. Cl. G01N 33/00

(52) U.S. Cl. 436/106

(57) ABSTRACT

Devices and methods for detecting the constituent parts of biological polymers are disclosed. A molecular analysis device includes a molecule sensor and a molecule guide. The molecule sensor comprises a nanostructure, which is configured for producing a nanostructure-enhanced Raman scattered radiation when an excitation radiation irradiates at least a portion of a molecule disposed near the NERS structure.

### FIELD OF THE INVENTION

The present invention relates to chemical analysis using nanoscale systems. More particularly, the present invention relates to systems for determining chemical sequences of biological polymers using nanoscale transport systems and nanostructure-enhanced Raman spectroscopy.

### BACKGROUND OF THE INVENTION

Determining the sequence of a Deoxyribonucleic acid (DNA) molecule is, conventionally, a difficult and expensive chemical process. However, with the rapid growth in nanotechnology new methods may be devised to increase accuracy, speed, and cost of determining the constituent parts of biological polymers such as proteins, DNA, and ribonucleic acid (RNA).

Various methods have been developed for determining the chemical composition of portions of a DNA strand or the chemical composition of an entire DNA strand. One such method involves creating a micro-array with hundreds or thousands of patches of single stranded DNA, which are often referred to as probes, attached to various locations on a substrate such as glass or silicon.

When using this DNA detection method, the DNA to be examined is first transcribed into RNA. RNA is a molecule very similar to DNA that can encode the same information as DNA. The RNA can then be used to create single stranded DNA (ssDNA) copies of the RNA. Fluorescent molecules, also referred to as tags, are then bonded onto the new single stranded DNA molecules.

When these tagged single stranded DNA molecules are washed over the micro-array, they will bond and stick to any of the single stranded DNA probes having a gene sequence with bases that are complementary to, but arranged in the same order as, the bases of the tags. Then, a light source exposing the micro-array causes the tagged DNA molecules that have stuck to the micro-array to fluoresce. The fluorescent glow can be detected and, based on where the various DNA tags were placed and their corresponding sequence, the sequence of the portion of the DNA stuck to that site can be determined.

Unfortunately, this process requires a significant number of chemical and optical steps to determine various portion of a DNA sequence. In addition, the detection is limited to the variety of DNA probes on the micro-array. Long probes, with a large number of sequences can detect a significant match, but it becomes difficult to place every possible variation of long probes on a single micro-array. On the other hand, short probes may be incapable of detecting a desired long sequence.

Another proposed detection method involves examining a polymerase chain reaction replication process. An RNA polymerase may attach to a DNA molecule and begin separating the DNA strand. The RNA polymerase then traverses along the DNA strand opening newer regions of the DNA strand and synthesizing an RNA strand matching the opened portions of the DNA. As the RNA polymerase traverses along the DNA, the portion of the DNA opened by the RNA polymerase closes down and re-bonds after leaving the RNA polymerase. In this detection method, the RNA polymerase is attached to an electronic device, such as a single electron transistor. Whenever the polymerase replication takes place, a charge variation may occur on the single electron transistor for each portion of the DNA molecule opened up by the RNA polymerase. By detecting these charge variations, the composition of the portion of the DNA molecule that is transcribed can be determined.

Unfortunately, the polymerase chain reaction method relies on the occurrence of this biological process of replication. In addition, the RNA polymerase replication only begins and ends at certain defined points of the DNA strand. As a result, it may be difficult to discover all portions of the DNA strand to be examined.

A device and method with the flexibility to examine the entire sequence of a DNA strand, without requiring complicated chemical processing is needed. A molecule detection system using nanoscale devices without the requirement of a biological replication process may be a smaller and less costly system than conventional approaches. This integrated molecule detection system may be easier to use and may be adaptable to detect a variety of predetermined sets of bases within DNA molecules.

### BRIEF SUMMARY OF THE INVENTION

The present invention, in a number of embodiments, includes molecular analysis devices and methods for detecting the constituent parts of biological polymers. An exemplary embodiment of a molecular analysis device comprises a molecule sensor and a molecule guide. The molecule sensor comprises a nanostructure (also referred to as a NERS structure), which is configured for producing a nanostructure-enhanced Raman scattered radiation when an excitation radiation irradiates at least a portion of a molecule disposed substantially near the NERS structure. The molecule guide is configured for guiding the molecule such that an identifiable configuration of the portion of the molecule is disposed sufficiently near the NERS structure to enable production of the nanostructure-enhanced Raman scattered radiation.

Another exemplary embodiment of a molecular analysis device comprises a molecule sensor and a molecule guide. The molecule sensor includes a NERS structure configured for producing a nanostructure-enhanced Raman scattered radiation when an excitation radiation irradiates at least a portion of a molecule disposed substantially near the NERS structure. The molecule sensor also includes a nitrogenous material disposed on the NERS structure, wherein the nitrogenous material is configured for a chemical reaction with an identifiable configuration of the molecule. The molecule guide is configured for guiding the identifiable configuration of the molecule substantially near the molecule sensor to enable the chemical reaction.

Another exemplary embodiment includes a method of analyzing a molecule. The method comprises guiding at least a portion of the molecule in a molecule guide such that an identifiable configuration of the molecule is disposed near a molecule sensor. The molecule sensor includes a NERS structure configured for producing a nanostructure-enhanced Raman scattered radiation. The method further includes irradiating the portion of the molecule disposed substantially near the NERS structure, with an excitation radiation. The method further includes detecting a Raman scattered radiation resulting from irradiation of the portion of the molecule and detecting the nanostructure-enhanced Raman scattered radiation resulting from irradiation of the identifiable configuration substantially near the NERS structure.

Yet another exemplary embodiment includes a method of analyzing a molecule. The method comprises guiding at least a portion of the molecule in a molecule guide such that an identifiable configuration of the molecule is disposed substantially near a molecule sensor. The molecule sensor includes a NERS structure configured for producing a nanostructure-enhanced Raman scattered radiation. The method further includes developing a chemical reaction between an identifiable configuration of the molecule and a nitrogenous material disposed on the NERS structure. The method further includes irradiating the portion of the molecule disposed substantially near the molecule sensor with an excitation radiation. The method further includes detecting a Raman scattered radiation resulting from irradiation of the portion of the molecule and detecting the nanostructure-enhanced Raman scattered radiation resulting from irradiation of the chemical reaction between the identifiable configuration of the molecule and the nitrogenous material.

### BRIEF DESCRIPTION OF THE SEVERAL VIEWS OF THE DRAWINGS

While the specification concludes with claims particularly pointing out and distinctly claiming that which is regarded as the present invention, the advantages of this invention can be more readily ascertained from the following description of the invention when read in conjunction with the accompanying drawings in which:

FIG. 1A is a three dimensional view of a portion of a DNA molecule;

FIG. 1B is a flat view of a portion of a DNA molecule showing various possible base pair bondings;

FIG. 2Ais a top view of an exemplary molecular analysis device including a nanochannel and one or more molecule sensors disposed in the nanochannel;

FIG. 2Bis a top view of an exemplary molecular analysis device including a plurality of nanochannels and molecule sensors disposed in the nanochannel;

FIGS. 3A, 3B, 3C, and 3D are three dimensional views of exemplary configurations of nanochannels useful in practicing the present invention;

FIG. 4A is a top view of an exemplary molecular analysis device including a nanopore and one or more molecule sensors;

FIG. 4B is a top view of an exemplary molecular analysis device including a plurality of nanopores and a plurality of molecule sensors;

FIG. 4C is a three dimensional view of an exemplary configuration of a nanopore and a molecule sensor;

FIG. 5 is a top view of an exemplary NERS structure and a nucleic acid chain;

FIG. 6 is a top view of another exemplary NERS structure including a base disposed thereon and a nucleic acid chain;

FIG. 7 is a top view of another exemplary NERS structure including an oligonucleotide disposed thereon and a nucleic acid chain;

FIG. 8 is a three dimensional view of an exemplary nanochannel including a NERS structure and a nucleic acid chain in the nanochannel; and

FIG. 9 is a block diagram of an exemplary molecular analysis system including a radiation source, a radiation detector, and a molecular analysis device.

### DETAILED DESCRIPTION OF THE INVENTION

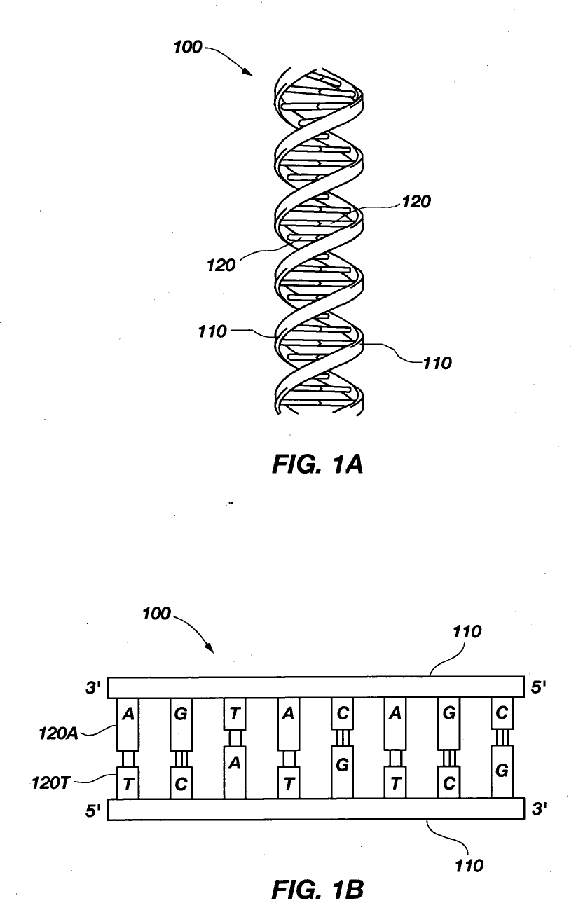

The present invention, in a number of embodiments, includes structures, devices, and methods for use in detecting the molecular structure of biological polymers. As illustrated in FIGS. 1A and 1B, an example of one such biological polymer is Deoxyribonucleic acid (DNA). A DNA molecule 100 comprises a double helix structure including two backbone strands 110 on the outside of the double helix. The backbone strands 110 are a structure made up of sugar-phosphate polymer strands. Between the two backbone strands 110 are pairs of bases 120 configured similar to ladder rungs. The bases 120 connecting the strands consist of four types: adenine 120A (A), thymine 120T (T), guanine 120G (G), and cytosine 120C (C). RNA, which is closely related to DNA, comprises a similar structure including the A, G, and C bases of DNA. However, in RNA, rather than bonding with T, A bonds with the molecule uracil (U) (not shown), which is closely related to T. In addition, while RNA can form a double helix, in nature it generally exists as a single strand.

Each of the base molecules 120 comprise nitrogenous compounds in various configurations. The base molecules 120 may bond with each other to form base pairs. As shown in FIG. 1B, T may form two weak hydrogen bonds with A, while C may form three weak hydrogen bonds with G. These weak bonds between the base pairs allow a DNA strand to be separated into two complementary single stranded molecules. A single human DNA molecule may include as many as three billion of these base pairs.

Another way of characterizing the constituent parts of a DNA strand is to consider the various bases 120 chemically bonded to a sugar. In this form, the resultant molecule is often referred to as a nucleoside. Each nucleoside includes a sugar molecule bonded to one of the various bases 120. A nucleoside with a phosphate molecule bonded to the sugar portion of the nucleoside is often referred to as a nucleotide. Thus, each strand of a DNA molecule may be considered as a plurality of nucleotides bonded together, wherein the bonds form at the sugar-phosphate portion of each nucleotide to form the backbone 110 of the strand. Nucleotides join together to form the backbone strands 110 by a 5′-3′ phosphodiester linkage, giving the strands a directionality. Thus, the 5′ end of the strand has a free phosphate group and the 3′ end has a free hydroxyl group. In double stranded DNA, the backbone strands 110 run in opposite directions such that each end of the double strand has a 5′ end on one backbone strand 110 and a 3′ end on the other backbone strand 110.

A section of single stranded DNA including a small plurality of nucleotides is often referred to as an oligonucleotide. These oligonucleotides are conventionally used as the tags in the prior art DNA micro-arrays previously described.

In genetic coding, an oligonucleotide comprising three consecutive nucleotides along RNA or single stranded DNA is often referred to as a codon. Any three consecutive nucleotides of A, C, G, and T (or U for RNA), can be combined in 64 (i.e., 4³) possible combinations. The 20 different amino acids are specified by these 64 different codons and are represented by more than one codon. For example, the amino acid Alanine may be represented by the codons GCA, GCC, GCG, and GCU.

Polypeptides and proteins (one or more polypeptide chains) are composed of a linear chain of amino acids covalently linked by peptide bonds. In addition to the codons that specify the various amino acids, some codons are defined as start codons and stop codons. These start and stop codons define the beginning and ending of the sequence of amino acids to be formed that ultimately form any given polypeptide or protein. Thus, identification of the various amino acids by direct identification of the 64 possible codons is possible.

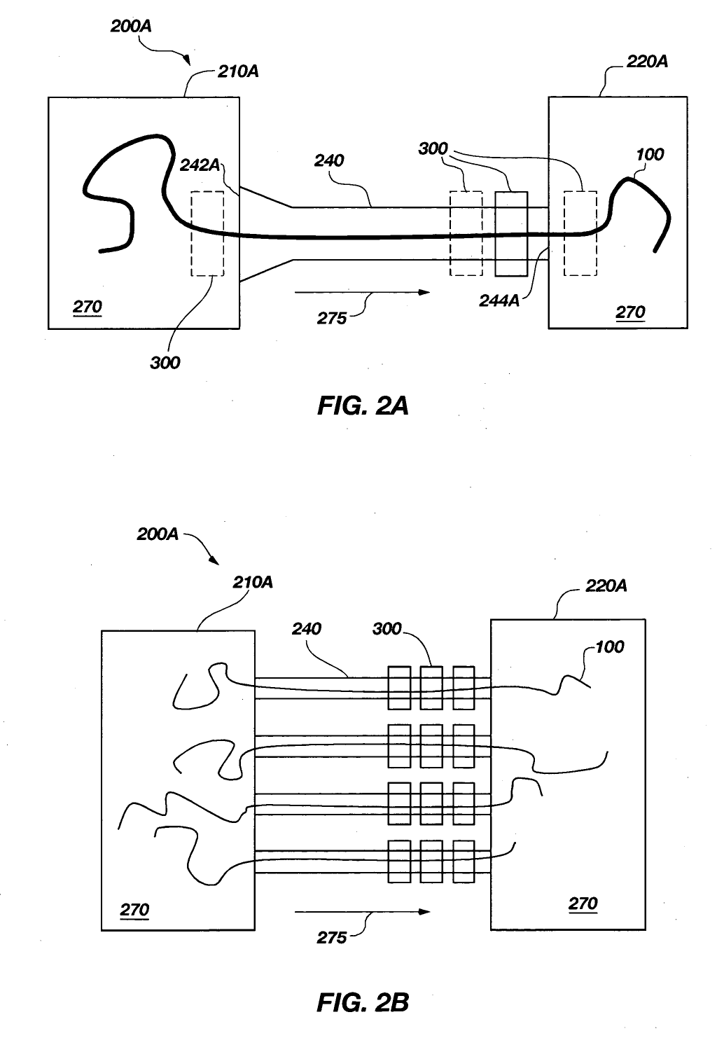

FIG. 2A illustrates an exemplary embodiment of a molecular analysis device 200A for analyzing biological polymers such as nucleic acid chains, including DNA and RNA. The molecular analysis device 200A includes a supply reservoir 210A, an accumulation reservoir 220A, a molecule guide (such as a nanochannel 240 shown in FIG. 2A), and at least one molecule sensor 300. In addition, a transport medium 270, such as, for example, an electrolyte solution, may be contained within the supply reservoir 210A, the nanochannel 240, and the accumulation reservoir 220A. At least one nucleic acid chain 100 may be disposed within the transport medium 270. The molecule sensor 300 is described in more detail below.

The nanochannel 240 may be configured as a nanofluidic channel for carrying the nucleic acid chain 100 in the transport medium 270 from the supply reservoir 210A, through the nanochannel 240, to the accumulation reservoir 220A in the transport direction 275 shown. Alternatively, the transport medium 270 may be configured for carrying the nucleic acid chain 100 from the accumulation reservoir 220A, through the nanochannel 240 to the supply reservoir 210A. Various methods may be used to transport the nucleic acid chain 100 through the nanochannel 240, such as, by way of example, electrokinetic flow, electroosmotic flow, hydrostatic pressure, hydrodynamic pressure, and hydromagnetic flow. These transport mechanisms may be caused by mechanical, magnetic, electrical field, heat-induced, and other methods known to a person of ordinary skill in the art.

Electrophoresis causes the movement of particles that are suspended in a medium to which an electromotive force is applied. Particularly, a particle or molecule having an electrical charge will experience an electromotive force when positioned within an electrical field. Nucleic acid chains 100 are good candidates for electrophoresis because they carry multiple negative charges due to the phosphate group and the phosphodiester backbone strand 110 (FIGS. 1A and 1B). Thus, when electrodes (not shown) with a voltage differential are placed in the transport medium 270, the nucleic acid chains 100 will migrate toward the more positive electrode. By way of example, if an electrode with a ground potential is placed in the supply reservoir 210A and an electrode with a positive voltage is placed in the accumulation reservoir 220A, nucleic acid chains 100 in the transport medium 270 will migrate from the supply reservoir 210A, through the nanochannel 240, and toward the electrode in the accumulation reservoir 220A. Furthermore, the movement rate or velocity of the nucleic acid chain 100 substantially correlates with the voltage bias between the electrodes. As a result, a first approximation of the nucleic acid chain 100 velocity may be determined, which may be used by, and refined by, signal processing analysis in combination with signal data from the molecule sensor 300 to determine the constituent parts of the nucleic acid chain 100.

Other transport mechanisms may rely on nanofluidic flow of the transport medium 270 itself, with the nucleic acid chain 100 being carried along with the transport medium 270. For example, electrokinetic flow (often referred to as electroosmotic flow) is generated in a similar manner to electrophoresis by electrodes (not shown) in the supply reservoir 210A and the accumulation reservoir 220A. Electrokinetic flow of the transport medium 270 may generally require higher voltage potentials to cause transport medium 270 flow than the voltage required to cause electrophoretic movement of the nucleic acid chains 100. Thus, nucleic acid chain 100 movement may be substantially electrophoretic or may be a combination of electrophoretic movement and movement caused by electrokinetic flow of the transport medium 270.

Yet another transport mechanism may rely on pressure driven flow. In very small channels, such as nanochannels 240, a small pressure differential may be developed by applying a temperature differential between the supply reservoir 210A and the accumulation reservoir 220A. This small pressure differential may cause the flow of the transport medium 270, and nucleic acid chains 100 within the transport medium 270, from one reservoir (210A, 220A) to the other reservoir (220A, 210A).

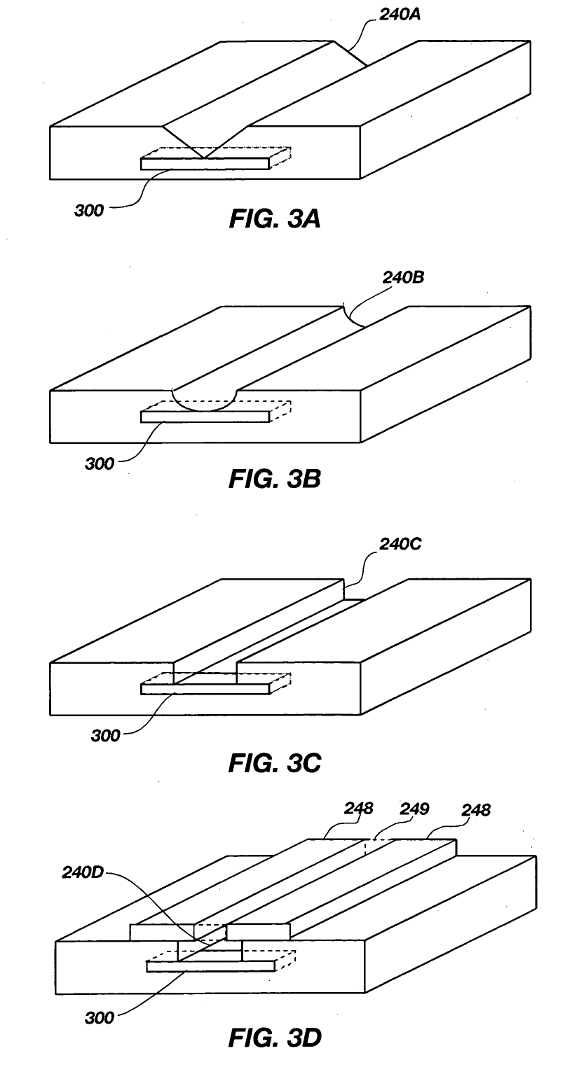

As shown in FIGS. 3A through 3D, the nanochannel 240 may be formed in a variety of configurations and cross sections. FIG. 3A illustrates a nanochannel 240A with a triangular cross section and a molecule sensor 300 positioned in the nanochannel 240A. FIG. 3B illustrates a nanochannel 240B with a semi-elliptical cross section and a molecule sensor 300 positioned in the nanochannel 240B. FIG. 3C illustrates a nanochannel 240C with a rectangular cross section and a molecule sensor 300 positioned in the nanochannel 240C. FIG. 3D illustrates a nanochannel 240D with a rectangular cross section and a molecule sensor 300 positioned in the nanochannel 240D. FIG. 3D further illustrates a partial channel cover 248 formed over a portion of the nanochannel 240 so that the nanochannel 240 is partially enclosed. Alternatively, a full channel cover 249 may be formed over the entire nanochannel 240 as shown by the dashed lines indicating a fully enclosed nanochannel 240.

Other nanochannel 240 cross sections are contemplated as being within the scope of the present invention, such as, by way of example and not limitation, circular, semi-circular, triangular, square, and hexagonal. Of course, the partially enclosed and fully enclosed nanochannel embodiments shown in FIG. 3D may be used with any of the various cross sections.

The nanochannels 240, partial channel covers 248, and full channel covers 249 may be fabricated using a variety of lithographic techniques, nano-imprint lithographic techniques, self-assembly techniques, template synthesis, wafer bonding, or combinations thereof. Additionally, the nanochannel 240 may be formed initially as a fully enclosed structure without the need for additional steps to form a partial channel cover 248 or full channel cover 249.

The length of the nanochannel 240 may vary from nanometers to orders of magnitude longer for adaptation to various applications and nucleic acid chain 100 lengths to be analyzed. Furthermore, the nanochannels 240 may include curves of a radius favorable to nucleic acid chain 100 flow and may be configured to enable long channels in a restricted area.

The nanochannel 240 is configured to at least partially straighten the nucleic acid chain 100 such that loops do not form within the channel and such that the nucleic acid chain 100 may be presented substantially near the molecule sensor 300. To ensure that loops do not form within the channel, in a particular embodiment, the channel cross section may need to be about twice the persistence length of the nucleic acid chain 100, or less. At room temperature, the persistence length for double stranded DNA is about 50 nm (i.e., L). Therefore, the nanochannel 240 should be about 100 nm (i.e., 2 L) or less to ensure that loops do not form.

To ensure that the nucleic acid chain 100 is presented substantially near the molecule sensor 300, the nanochannel 240 may need to be significantly narrower than the width needed to keep the nucleic acid chain 100 from forming loops. Thus, nanochannel 240 cross section dimensions may vary depending on the type of molecule sensor 300 used, as explained more fully below in the discussion of the molecule sensor 300. Furthermore, the cross section dimensions may vary along the length of the nanochannel 240. For example, a nanochannel 240 may have a relatively wide cross section for much of its length and narrow down to a smaller cross section near a molecule sensor 300.

Returning to FIG. 2A, a molecule sensor 300 is shown in the nanochannel 240 near an exit point 244A of the nanochannel 240. Other optional molecule sensors 300 are also shown to illustrate the flexibility and possibilities for positioning of the molecule sensors 300 relative to the nanochannel 240 and nucleic acid chain 100. It may be desirable to place multiple molecule sensors 300 in various positions to detect various portions of the nucleic acid chain 100. For example, an optional molecule sensor 300 is shown in the nanochannel 240, an optional molecule sensor 300 is shown in the supply reservoir 210A substantially near an entrance point 242A of the nanochannel 240, and an optional molecule sensor 300 is shown in the accumulation reservoir 220A substantially near the exit point 244A of the nanochannel 240. Molecule sensors 300 outside of the nanochannel 240 (i.e., near the entrance point 242A or exit point 244A) may be placed in a location where the nucleic acid chain 100 is still presented substantially near the molecule sensors 300 and where the nucleic acid chain 100 has not assumed an un-straightened configuration. It will be understood by those of ordinary skill in the art that the labeling of entrance point 242A and exit point 244A are arbitrary, as the molecular analysis device 200A may be configured to cause flow of the nucleic acid chain 100 in either direction through the nanochannel 240.

FIG. 2B illustrates a plurality of nanochannels 240 all coupled to a single supply reservoir 210A and a single accumulation reservoir 220A, with a nucleic acid chain 100 in each of the plurality of nanochannel 240. In addition, each of the nanochannels 240 is shown with a plurality of molecule sensors 300 in the nanochannels 240 and a transport direction 275 from the supply reservoir 210A to the accumulation reservoir 220A. A person of ordinary skill in the art will appreciate that many configurations of reservoirs (210A, 220A), nanochannels 240, and molecule sensors 300 are contemplated within the scope of the invention.

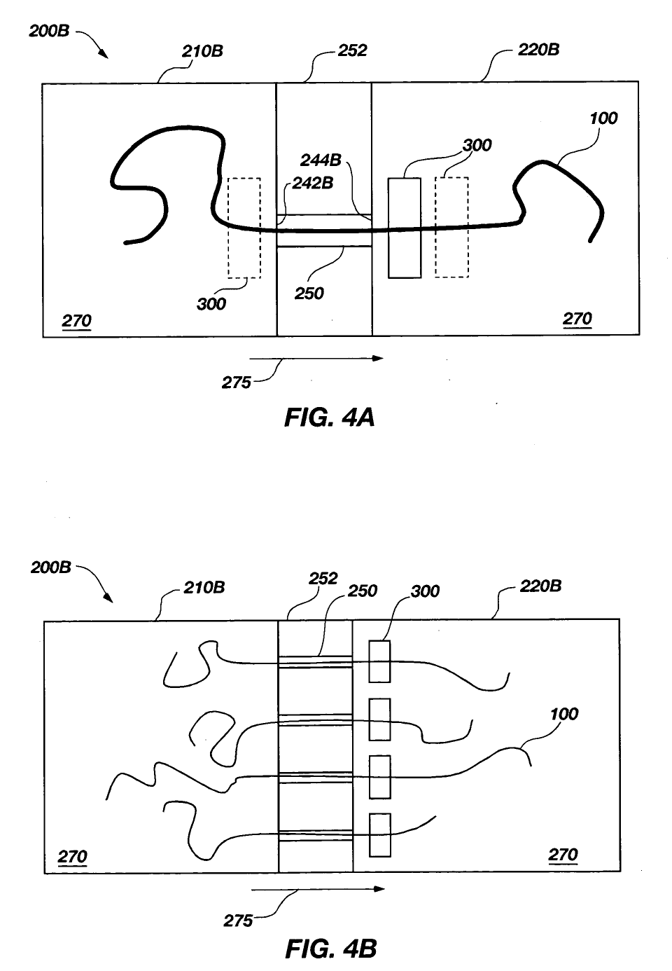

FIG. 4A illustrates another exemplary embodiment of a molecular analysis device 200B for analyzing biological polymers. The molecular analysis device 200B includes a supply reservoir 210B, an accumulation reservoir 220B, a molecule guide (also referred to as a nanopore 250 in the embodiment of FIG. 4A), and a molecule sensor 300. In addition, a transport medium 270, such as, for example, an electrolyte solution, may be contained within the supply reservoir 210B, the nanopore 250, and the accumulation reservoir 220B. At least one nucleic acid chain 100 may be disposed within the transport medium 270. The molecule sensor 300 is described in more detail below.

The nanopore 250 may be configured for carrying the nucleic acid chain 100 in the transport medium 270 from the supply reservoir 210B, through the nanopore 250, to the accumulation reservoir 220B in the transport direction 275 shown. Alternatively, the transport medium 270 may be configured for carrying the nucleic acid chain 100 from the accumulation reservoir 220B, through the nanopore 250, to the supply reservoir 210B. The same methods discussed above for transportation of the nucleic acid chain 100 through the nanochannel 240 of FIGS. 1 and 2 are applicable for transportation of the nucleic acid chain 100 through the nanopore 250.

A nanopore 250, as shown in FIGS. 4A, 4B, and 4C has an opening of from about 1 nanometer to about 100 nanometers, in a membrane 252. The membrane 252 may comprise an organic or inorganic material, which may be fabricated using a variety of lithographic techniques, nano-imprint lithographic techniques, self-assembly techniques, template synthesis, wafer bonding, or combinations thereof.

The nanopore 250 may be cylindrical in shape (as shown in FIG. 4C) or may include other cross sectional shapes such as, by way of example only, triangular, square, hexagonal, and octagonal. The figures illustrating nanopores 250 in membranes 252 are generally shown with a nanopore 250 configured horizontally through a vertical membrane 252. However, the membrane 252 may be disposed horizontally, with a vertical nanopore 250 therethrough, or any other suitable configuration, so long as the nanopore 250 may be configured to present successive segments of the nucleic acid chain 100 substantially near the molecule sensor 300, as explained below.

In a particular embodiment, the nanopore 250 may be about 100 nm or less to ensure the nucleic acid chain 100 does not pass through the nanopore 250 in some type of looped configuration, as explained above in the discussion of persistence length. To ensure that the nucleic acid chain 100 is presented substantially near the molecule sensor 300, the nanopore 250 may need to be significantly narrower than the width needed to keep the nucleic acid chain 100 from forming loops. Thus, nanochannel 240 cross section dimensions may vary depending on the type of molecule sensor 300 used, as explained more fully below in the discussion of the molecule sensor 300.

The membrane 252 may be a wide variety of thicknesses because the invention uses the nanopore 250 as a presentation and transport mechanism, rather than a sensing mechanism. A relatively thin membrane 252 may enable more uniform nanopores 250. A relatively thick membrane 252 may assist in straightening the nucleic acid chain 100 in the vicinities of the nanopore 250 entrance point 242B and nanopore 250 exit point 244B, allowing additional molecule sensors 300 to be lined up in the area where the nucleic acid chain 100 remains relatively straight such that it can be transported substantially close to a plurality of molecule sensors 300.

In FIG. 4A, a molecule sensor 300 is shown substantially near an exit point 244B of the nanopore 250. Other optional molecule sensors 300 are also shown to illustrate the flexibility and possibilities for positioning of the molecule sensors 300 relative to the nanopore 250 and nucleic acid chain 100. It may be desirable to place multiple molecule sensors 300 in positions to detect various portions of the nucleic acid chain 100. As examples, an optional molecule sensor 300 is shown in the supply reservoir 210B substantially near an entrance point 242B of the nanopore 250, and an additional molecule sensor 300 is shown in the accumulation reservoir 220B near the exit point 244B of the nanopore 250. Molecule sensors 300 near the entrance point 242B or exit point 244B may be placed in a location where the nucleic acid chain 100 is presented substantially near the molecule and wherein the nucleic acid chain 100 has not assumed its folded (un-straightened) configuration. It will be understood by those of ordinary skill in the art that the labeling of entrance point 242B and exit point 244B are arbitrary, as the molecular analysis device 200 may be configured to cause flow of the nucleic acid chain 100 in either direction through the nanopore 250.

FIG. 4B illustrates a plurality of nanopores 250 all coupled to a single supply reservoir 210A and a single accumulation reservoir 220B, with a nucleic acid chain 100 in each of the plurality of nanopores 250 and a transport direction 275 from the supply reservoir 210B to the accumulation reservoir 220B. A person of ordinary skill in the art will appreciate that many configurations of reservoirs (210B, 220B), nanopores 250, and molecule sensors 300 are contemplated within the scope of the invention.

The present invention also utilizes Raman Spectroscopy structures and, more specifically, Nanostructure-Enhanced Raman Spectroscopy structures, as detailed below. Raman Spectroscopy is a well-known spectroscopic technique for performing chemical analysis of an analyte in the gas, liquid or solid phase. In conventional Raman Spectroscopy, high intensity monochromatic light from a light source, such as a laser, is directed onto an analyte to be chemically analyzed. The analyte may contain a single species of molecules or mixtures of different molecules. Furthermore, Raman Spectroscopy may be performed on a number of different molecular configurations, such as organic and inorganic molecules in crystalline or amorphous states.

For a system that does not have surface or nanostructure enhancement, the majority of incident photons of the light are elastically scattered by the analyte molecule. In other words, the scattered photons have the same frequency, and thus the same energy, as the photons that were incident on the analyte. This is known as Rayleigh scattering. However, a small fraction of the photons (i.e., 1 in 10⁷ photons) are inelastically scattered by the un-enhanced analyte molecule at a different optical frequency than the incident photons. The inelastically scattered photons are termed “Raman scattered radiation” and may be scattered at frequencies greater than, but most are usually scattered at a frequency lower than, the frequency of the incident photons. When the incident photons collide with the analyte and give up some of their energy, the Raman scattered photons emerge with a lower energy and thus at a lower frequency. The lower energy Raman scattered photons are commonly referred to in Raman spectroscopy as the “Stokes radiation.” A small fraction of the molecules are already in an energetically excited state and when the incident photons collide with the molecules, the Raman scattered photons emerge at a higher energy and thus at a higher frequency. The higher energy Raman scattered photons are commonly referred to in Raman spectroscopy as the “anti-Stokes radiation.” Raman scattering may occur from the rotational, vibrational, or electronic states of the molecules.

By analyzing the intensity of the inelastically scattered Raman photons against frequency, a unique Raman spectrum, which corresponds to the particular analyte molecule, may be obtained. This Raman spectrum may be used to identify chemical species, as well as other physical properties of the analyte. While conventional Raman Spectroscopy is suitable for bulk chemical analysis, it is not effective for surface studies because the signal from the bulk Raman scattered photons overwhelms any signal from Raman scattered photons near the surface.

Due to the deficiencies with performing surface studies using conventional Raman Spectroscopy, another Raman Spectroscopy technique called Surface Enhanced Raman Spectroscopy (SERS), which is effective for performing surface studies, has been developed. In SERS, a monolayer, or sub-monolayer, amount of the molecules to be analyzed is adsorbed onto a specially roughened metal surface. Typically, the metal surface is made from gold, silver, copper, or semiconducting/dielectric material which include Si, GaAs, GaN, diamond, InP, carbon, ZnSe, ZnO and all its alloys. Raman Spectroscopy has also been used employing nanostructures, such as, for example, metallic nanoparticles or nanowires disposed on a surface, as opposed to a roughened metallic surface. Enhancement of Raman spectroscopy using nanostructures is hereinafter referred to as Nanostructure-Enhanced Raman Spectroscopy (NERS). Similarly, structures including nanostructures suitable for enhancing Raman Spectroscopy are referred to as NERS structures. Thus, Raman spectroscopy may be used to study monolayers of materials adsorbed on metals, and even single molecules adsorbed near an appropriate nanostructure (i.e., a NERS structure).

In hyper-Raman spectroscopy, when excitation radiation impinges on an analyte molecule, a very small number of photons may be scattered at frequencies corresponding to the higher order harmonics of the excitation radiation, such as the second and third harmonics (i.e., twice or three times the frequency of the excitation radiation). Some of these photons may be Raman scattered photons with a frequency that is Raman-shifted relative to the frequencies corresponding to the higher order harmonics of the excitation radiation. Therefore, in hyper-Raman spectroscopy, the incident excitation photons have approximately ½, ⅓, or ¼ the frequency of the Raman photons.

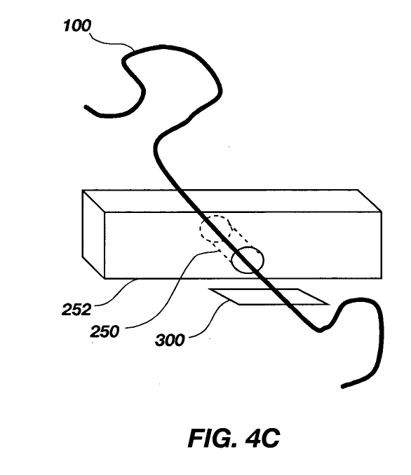

FIG. 5 illustrates an exemplary NERS structure 310, which includes a plurality of nanoparticles 320 disposed on a suitable substrate. The NERS structure 310 is shown in the figures as a dashed line. The dashed line does not necessarily indicate a specific structure. Rather, the dashed line just indicates a boundary of the plurality of nanoparticles 320 comprising the NERS structure 310. A nucleic acid chain 100 is shown disposed near at least two of the plurality of nanoparticles 320. As used herein, the term “NERS structure” means any nanostructure configured and formed of a material that may produce chemical enhancement of the Raman signal, electromagnetic enhancement of the Raman signal, or both. Exemplary materials for formation of the nanostructure are metallic materials, which include gold, silver, copper, aluminum, chromium, or semiconducting/dielectric material which include Si, GaAs, GaN, diamond, InP, carbon, ZnSe, ZnO and all its alloys. However, any other suitable material that may produce chemical enhancement of the Raman signal, electromagnetic enhancement of the Raman signal, or both, is contemplated as being within the scope of the present invention.

As one or more discrete nanoparticles 320, the NERS structure 310 may have a variety of exemplary configurations, such as, for example, nanodots and nanoparticles. In one particular embodiment of the invention, at least two silver nanoparticles 320 spaced apart from one another, such that an identifiable configuration of the nucleic acid chain 100 may be draped therebetween. The nanostructures for the NERS structure 310 may be formed by chemical vapor deposition (CVD), molecular beam epitaxy (MBE), atomic layer deposition (ALD), nano-imprint lithography, or any other known technique for depositing the NERS structure 310 on a substrate.

The substrate (not shown) may be any suitable substrate for integrating the NERS structure 310 and the molecule guide. In addition, if optional components are present, the substrate may be selected for its properties of directing and modifying either optical signals, electrical signals, or both optical signals and electrical signals. By way of example, conventional silicon substrates, as well as Group III-V materials, such as gallium arsenide, and indium phosphide may be desirable.

In the embodiment of FIG. 5, an excitation radiation 360 illuminates the NERS structure 310 and an identifiable configuration of the nucleic acid chain 100. This illumination may generate a nanostructure-enhanced Raman scattered radiation 370 unique to the identifiable configuration. When evaluating nucleic acid chains, depending on the size of the nanoparticles 320, the identifiable configuration may comprise a plurality of bases. By way of example, and not limitation, if the nanoparticles 320 have a size of about four nanometers, there may be as many as ten to fifteen bases substantially near the nanoparticles 320 at any given time. Thus as the nucleic acid chain 100 moves across the NERS structure 310, the nanostructure-enhanced Raman scattered radiation 370 will change depending on the bases that are substantially near the NERS structure 310 at that time. The nanostructure-enhanced Raman scattered radiation 370, and its change over time as the nucleic acid chain 100 moves over the NERS structure 310, may be detected and analyzed to determine the sequence of individual bases in the nucleic acid chain 100. This detection and analysis is explained more fully below.

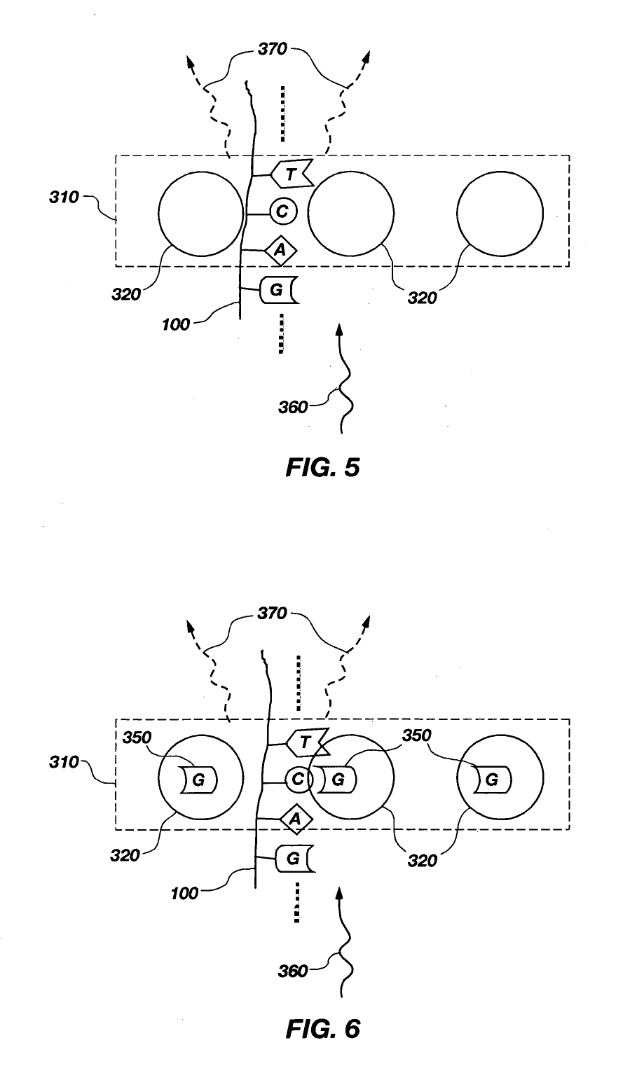

FIG. 6 illustrates another exemplary NERS structure 310, which includes a nitrogenous material 350 disposed on the nanoparticles 320. The nitrogenous material 350 may comprise a base 120 selected from the group consisting of adenine 120A, thymine 120T, uracil 120U, cytosine 120C, and guanine 120G. Furthermore, the nitrogenous material 350 coating the nanoparticle 320 may also include a sugar bonded to the base 120 or a sugar-phosphate bonded to the base 120. By way of example, FIG. 6 illustrates the nitrogenous material 350 guanine 120G. Guanine 120G is illustrated in FIG. 6 as a representative molecule to show functional interaction with the nucleic acid chain 100. However, generally, the entire NERS structure 310 may be coated with the nitrogenous material 350.

As the nucleic acid chain 100 passes substantially near the coated NERS structure 310, a base 120 (in this example, C) of the nucleic acid chain 100 that is complementary to the nitrogenous material 350 (in this example, G) on the NERS structure 310 may react with the nitrogenous material 350. This reaction may take the form of a transitory chemical bond between the complementary base on the nucleic acid chain 100 and the nitrogenous material 350 on the NERS structure 310. The transitory chemical bond may cause a change in the nanostructure-enhanced Raman scattered radiation 370, which will aid in detecting the specific base 120 currently bonded to the nitrogenous material 350. In the FIG. 6 embodiment, an excitation radiation 360 illuminates the NERS structure 310 and the identifiable configuration of the nucleic acid chain 100 that is substantially near the NERS structure 310. This illumination may generate a nanostructure-enhanced Raman scattered radiation 370 unique to the identifiable configuration and the transitory chemical bond.

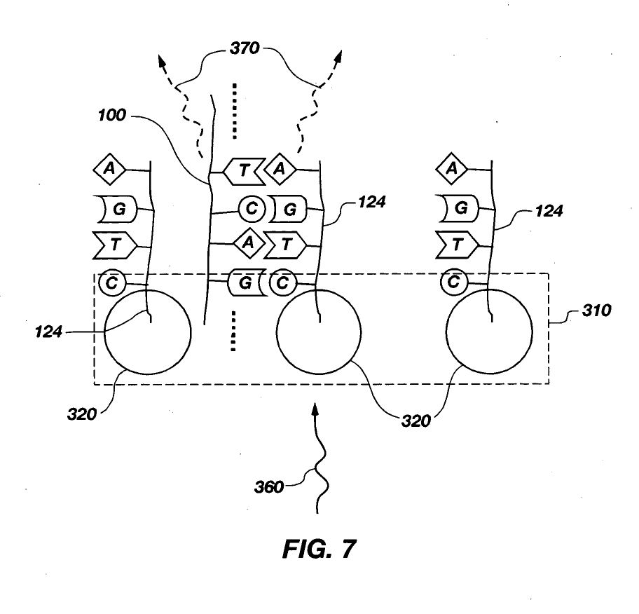

FIG. 7 illustrates another exemplary NERS structure 310 wherein the nitrogenous material 350 comprises an oligonucleotide 124 attached to the nanoparticles 320. The oligonucleotide 124 may include many combinations of nucleotides and may have various lengths to comprise a specific combination of nucleotides that may be of interest. By way of example, FIG. 7 illustrates an oligonucleotide 124 including four nucleotides in the series of C, T, G, and A. The attachment of the oligonucleotide 124 to the nanoparticles 320 may be accomplished with a variety of methods know to those of ordinary skill in the art, such as the methods used in micro-arrays using fluorescent tags.

As the nucleic acid chain 100 passes substantially near the attached oligonucleotide 124, if a complementary sequence of bases passes substantially near the attached oligonucleotide 124, a transitory chemical bond (i.e., hybridization) may occur between the oligonucleotide 124 and the complementary sequence on the nucleic acid chain 100. In the FIG. 7 exemplary embodiment, the oligonucleotide 124 comprising the sequence C, T, G, A, may hybridize with the complementary sequence G, A, C, T on the nucleic acid chain 100. As with the single base example of FIG. 6, this transitory chemical bond between the nucleic acid chain 100 and the attached oligonucleotide 124 will cause a different nanostructure-enhanced Raman scattered radiation 370 due to the different chemical makeup of the nucleic acid chain 100 substantially near the NERS structure 310 and the transitory chemical bond. Using this oligonucleotide embodiment, a plurality of molecule sensors 300 configured with a variety of oligonucleotides 124 may be useful in determining different specific characteristics of any given nucleic acid chain 100.

The transitory chemical bond to a single base 120 or an oligonucleotide 124 results from weak hydrogen bonds between the base 120 (or oligonucleotide 124) on the NERS structure 310 and the nucleic acid chain 100. The transitory chemical bond may be broken, allowing continued transportation of the nucleic acid chain 100, by the motive force causing transportation of the nucleic acid chain 100, thermal energy, optical energy, or combinations thereof.

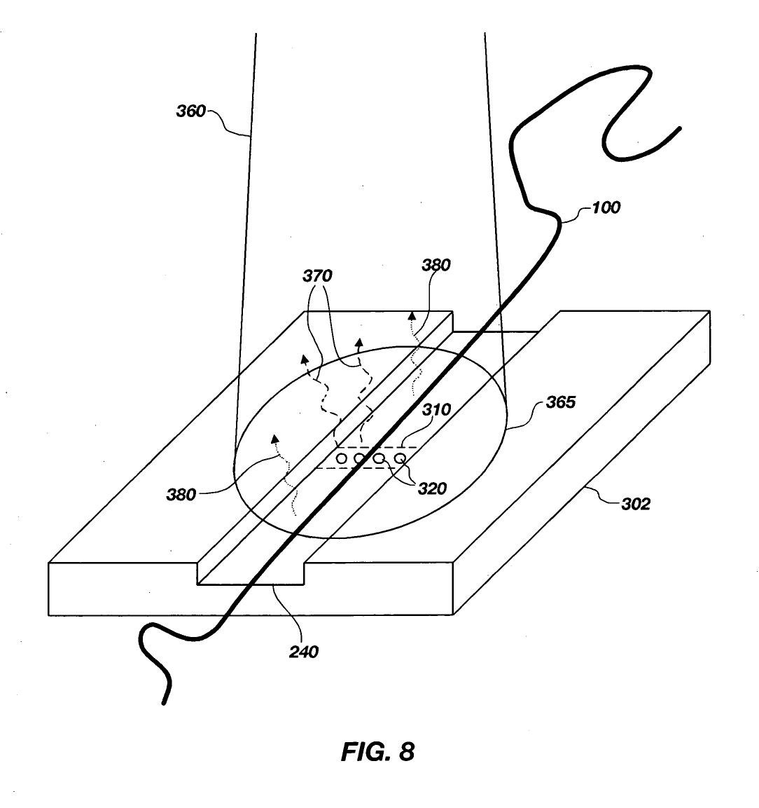

FIG. 8 illustrates an exemplary NERS structure 310 disposed in a nanochannel 240 located on a substrate 302. The excitation radiation 360 illuminates a portion of the nucleic acid chain 100. A Raman scattered radiation 380 may be generated which corresponds to the portion of the nucleic acid chain 100 within the area of illumination 365 created by the excitation radiation 360. In addition to the Raman scattered radiation 380, a nanostructure-enhanced Raman scattered radiation 370 may be generated, which is unique to the identifiable configuration substantially near the NERS structure 310. Furthermore, for the embodiments represented in FIGS. 6 and 7, the nanostructure-enhanced Raman scattered radiation 370 may be influenced by the transitory chemical bond between the nitrogenous material 350 and the nucleic acid chain 100.

With current laser technology, the area of illumination 365 may be as small as about 300 nanometers. However, areas of illumination 365 having different sizes are contemplated as being within the scope of the present invention, as the amount of nucleic acid chain 100 which is illuminated at any given time may be compensated for in the spectral analysis of the nanostructure-enhanced Raman scattered radiation 370 relative to the Raman scattered radiation 380.

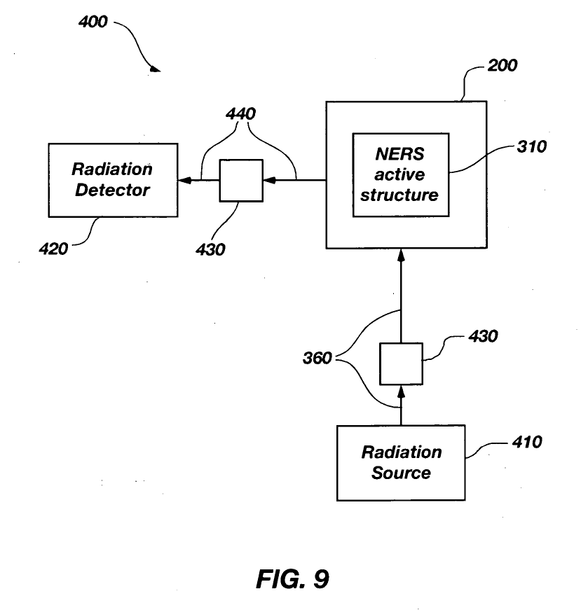

FIG. 9 illustrates, in block diagram form, an exemplary nanostructure-enhanced Raman scattered radiation system 400. The NERS system 400 includes a molecular analysis device 200 with a NERS structure 310 disposed thereon, a radiation source 410, and a radiation detector 420. The NERS system 400 may also include various optical components 430, such as, for example, lenses and filters positioned between the radiation source 410 and the molecular analysis device 200, and/or positioned between the molecular analysis device 200 and the radiation detector 420.

The radiation source 410 may include any suitable source for emitting excitation radiation 360 at the desired wavelength and may be capable of emitting a tunable wavelength of radiation. For example, commercially available semiconductor lasers, helium-neon lasers, carbon dioxide lasers, radiation-emitting diodes, incandescent lamps, and many other known radiation-emitting sources may be used as the radiation source 410. The wavelengths that are emitted by the radiation source 410 may include a suitable wavelength for performing NERS on the analyte molecule. Furthermore, the emitted radiation may be filtered by optical components 430 so that the proper wavelength is incident on the NERS structure 310, even where a broader spectrum of radiation is emitted by the radiation source 410. An exemplary range of wavelengths that may be useful in performing NERS includes wavelengths between about 350 nanometers and about 1000 nanometers.

The molecular analysis device 200 may also be used for hyper-Raman spectroscopy. When excitation radiation 360 impinges on an analyte molecule on a NERS structure 310, a small number of photons may be scattered at frequencies corresponding to the higher order harmonics of the excitation radiation 360, such as the second and third harmonics (i.e., twice or three times the frequency of the excitation radiation 360). Some of these photons may be Raman scattered photons with a frequency that is Raman-shifted relative to the frequencies corresponding to the higher order harmonics of the excitation radiation 360. Therefore, in hyper-Raman spectroscopy, as examples and not limitations of higher order harmonics, the incident excitation photons may have approximately ½, ⅓, or ¼ the frequency of the Raman photons.

With hyper-Raman excitation, the excitation frequency and Rayleigh scattered radiation at the excitation frequency may be filtered out from the Raman signal frequency by an inexpensive optical filter 430. In the present invention, NERS includes Raman, hyper-Raman and higher multiple excitation photons.

By way of example and not limitation, the excitation radiation 360 may have a wavelength of about 980 nm, and the Raman signal may have wavelengths of about 490 nm. As a result, the optical filter 430 may be configured to substantially filter out the radiation at and near the 980 nm wavelength, while allowing shorter wavelengths to pass through.

The detector receives and detects scattered radiation 440, which may include Raman scattered radiation 380 and nanostructure-enhanced Raman scattered radiation 370. The detector may include devices for determining the wavelength of the detected radiation (e.g., a monochromator) and devices for determining the intensity of the detected radiation (e.g., a photomultiplier). Generally, the scattered radiation 440 may scatter in all directions relative to the molecular analysis device 200. Thus, the detector may be positioned relative to the molecular analysis device 200 in a variety of locations. However, the detector may be positioned at, for example, an angle of about 90° relative to the direction of the excitation radiation 360 to minimize the intensity of any excitation radiation 360 that is incident on the detector.

Optical components 430 positioned between the radiation source 410 and the molecular analysis device 200 may be used to collimate, filter, or focus the excitation radiation 360 before the excitation radiation 360 impinges on the molecular analysis device 200 and the NERS structure 310. Optical components 430 positioned between the molecular analysis device 200 and the radiation detector 420 may be used to collimate, filter, or focus the scattered radiation 440. For example, a filter or a plurality of filters may be employed to prevent radiation at wavelengths corresponding to the excitation radiation 360 from impinging on the detector, thus allowing only the scattered radiation 440 to be received by the detector.

To analyze a molecule using the NERS system 400, the molecule may be transported through the molecule guide (not shown in FIG. 9) and presented to the NERS structure 310. The NERS structure 310 and analyte molecule are irradiated with excitation radiation 360 provided by the source. Scattered radiation 440, including Raman scattered radiation 380 and nanostructure-enhanced Raman scattered radiation 370, is detected by the detector. Thus, a complete Raman spectrum may be obtained for the portion of the molecule in the area of illumination 365 (shown in FIG. 8), which includes a Raman spectrum for the portion of the nucleic acid chain 100 illuminated and a nanostructure-enhanced Raman spectrum for the identifiable configuration substantially near the NERS structure 310.

Signal processing hardware, software, or a combination thereof may then be used to process the changes in the Raman spectrum as the molecule is transported through the area of illumination 365 (shown in FIG. 8) and across the NERS structure 310 relative to the speed of the nucleic acid chain 100. Thus, a complete identification of the nucleic acid chain 100 may be derived based on the velocity of the nucleic acid chain 100 and the relative Raman spectrum at any given time.

Although the foregoing description contains many specifics, these are not to be construed as limiting the scope of the present invention, but merely as providing certain exemplary embodiments. Similarly, other embodiments of the invention may be devised which do not depart from the spirit or scope of the present invention. The scope of the invention is, therefore, indicated and limited only by the appended claims and their legal equivalents, rather than by the foregoing description. All additions, deletions, and modifications to the invention, as disclosed herein, which fall within the meaning and scope of the claims, are encompassed by the present invention.

What is claimed is:

1. A molecular analysis device, comprising:
a molecule sensor comprising a NERS structure and configured for producing a Raman scattered radiation when an excitation radiation irradiates at least a portion of a molecule located near the molecule sensor; and
a molecule guide configured for guiding an identifiable configuration of the molecule sufficiently near the NERS structure to enable production of a nanostructure-enhanced Raman scattered radiation when the excitation radiation irradiates the identifiable configuration of the molecule.

2. The device of claim 1, wherein the molecule guide comprises:
a nanochannel including an entrance point and an exit point, the nanochannel configured for substantially straightening the molecule and guiding the molecule substantially near the NERS structure;
a transport medium disposed in the nanochannel and configured for transporting the molecule in a lengthwise fashion through the nanochannel in a direction from the entrance point to the exit point to successively present each segment of a plurality of segments distributed along the length of the molecule to the NERS structure.

3. The device of claim 2, wherein the transport medium near the exit point is positively charged relative to the transport medium near the entrance point.

4. The device of claim 2, wherein the NERS structure is positioned at a location selected from the group consisting of: substantially in the nanochannel between the entrance point and the exit point; external to the nanochannel and substantially near the entrance point of the nanochannel; and external to the nanochannel and substantially near the exit point of the nanochannel.

5. The device of claim 1, wherein the molecule guide comprises:
a nanopore formed in a membrane, the nanopore including an entrance point and an exit point and configured for guiding the molecule near the NERS structure; and
a transport medium configured for transporting the molecule through the nanopore to successively present each segment of a plurality of segments distributed along the length of the molecule to the NERS structure.

6. The device of claim 5, wherein the transport medium near the exit point is positively charged relative to the transport medium near the entrance point.

7. The device of claim 5, wherein the NERS structure is positioned at a location selected from the group consisting of: substantially near the entrance point of the nanopore; and substantially near the exit point of the nanopore.

8. The device of claim 1, wherein the excitation radiation comprises a wavelength that is an integer multiple of a wavelength near the nanostructure-enhanced Raman scattered radiation.

9. The device of claim 1, wherein the NERS structure comprises a plurality of nanoparticles, each nanoparticle of the plurality including a metallic material.

10. The device of claim 9, wherein the metallic material is selected from the group consisting of gold, silver, copper, aluminum, chromium, lithium, sodium, and potassium.

11. The device of claim 1, further comprising:
a radiation source configured for generating the excitation radiation; and
a radiation detector configured for detecting the nanostructure-enhanced Raman scattered radiation and the Raman scattered radiation.

12. The device of claim 11, further comprising an optical filter disposed between the molecule sensor and the radiation detector, the optical filter configured for substantially filtering out radiation near a wavelength of the excitation radiation.

13. A molecular analysis device, comprising:
a molecule sensor configured for producing a Raman scattered radiation when an excitation radiation irradiates at least a portion of a molecule near the molecule sensor, the molecule sensor comprising:
a NERS structure; and
a nitrogenous material disposed on the NERS structure, the nitrogenous material configured to react with an identifiable configuration of the molecule; and
a molecule guide configured for guiding the identifiable configuration of the molecule sufficiently near the molecule sensor to enable the reaction and production of a nanostructure-enhanced Raman scattered radiation when the excitation radiation irradiates the identifiable configuration.

14. The device of claim 13, wherein the nitrogenous material is configured to allow a transitory chemical bond between the nitrogenous material and the identifiable configuration of the molecule near the molecule sensor.

15. The device of claim 13, wherein the identifiable configuration of the molecule comprises a base selected from the group consisting of adenine, thymine, uracil, cytosine, and guanine.

16. The device of claim 13, wherein the nitrogenous material comprises a base selected from the group consisting of adenine, thymine, uracil, cytosine, and guanine.

17. The device of claim 16, wherein the nitrogenous material further comprises a material selected from the group consisting of a sugar chemically bonded to the base and a sugar-phosphate chemically bonded to the base.

18. The device of claim 13, wherein the nitrogenous material comprises an oligonucleotide and the identifiable configuration of the molecule is a complementary match to the oligonucleotide.

19. The device of claim 13, wherein the molecule guide comprises:
a nanochannel including an entrance point and an exit point, the nanochannel configured for substantially straightening the molecule and guiding the molecule substantially near the nitrogenous material;
a transport medium disposed in the nanochannel and configured for transporting the molecule in a lengthwise fashion through the nanochannel in a direction from the entrance point to the exit point to successively present each segment of a plurality of segments distributed along the length of the molecule to the nitrogenous material.

20. The device of claim 19, wherein the transport medium near the exit point is positively charged relative to the transport medium near the entrance point.

21. The device of claim 19, wherein the NERS structure is positioned at a location selected from the group consisting of: substantially in the nanochannel between the entrance point and the exit point; external to the nanochannel and substantially near the entrance point of the nanochannel; and external to the nanochannel and substantially near the exit point of the nanochannel.

22. The device of claim 13, wherein the molecule guide comprises:
a nanopore formed in a membrane, the nanopore including an entrance point and an exit point and configured for guiding the molecule near the nitrogenous material; and
a transport medium configured for transporting the molecule through the nanopore to successively present each segment of a plurality of segments distributed along the length of the molecule to the nitrogenous material.

23. The device of claim 22, wherein the transport medium near the exit point is positively charged relative to the transport medium near the entrance point.

24. The device of claim 22, wherein the NERS structure is positioned at a location selected from the group consisting of substantially near the entrance point of the nanopore and substantially near the exit point of the nanopore.

25. The device of claim 13, wherein the excitation radiation comprises a wavelength that is an integer multiple of a wavelength near the nanostructure-enhanced Raman scattered radiation.

26. The device of claim 13, wherein the NERS structure comprises a plurality of nanoparticles, each nanoparticle of the plurality including a metallic material.

27. The device of claim 26, wherein the metallic material is selected from the group consisting of gold, silver, copper, aluminum, chromium, lithium, sodium, and potassium.

28. The device of claim 13, further comprising:
a radiation source configured for generating the excitation radiation; and
a radiation detector configured for detecting the nanostructure-enhanced Raman scattered radiation and the Raman scattered radiation.

29. The device of claim 28, further comprising an optical filter disposed between the molecule sensor and the radiation detector, the optical filter configured for substantially filtering out radiation substantially near a wavelength of the excitation radiation.

30. A method of analyzing a molecule, comprising:
guiding at least a portion of the molecule in a molecule guide such that an identifiable configuration of the molecule is disposed substantially near a molecule sensor, the molecule sensor including a NERS structure;
irradiating the at least a portion of the molecule disposed near the NERS structure with an excitation radiation;
detecting a Raman scattered radiation resulting from irradiation of the at least a portion of the molecule; and
detecting a nanostructure-enhanced Raman scattered radiation resulting from irradiation of the identifiable configuration near the NERS structure.

31. The method of claim 30, further comprising analyzing the Raman scattered radiation and the nanostructure-enhanced Raman scattered radiation to determine a composition of the identifiable configuration.

32. The method of claim 30, further comprising generating a wavelength of the excitation radiation that is an integer multiple of a wavelength near the nanostructure-enhanced Raman scattered radiation.

33. The method of claim 30, wherein guiding at least a portion of the molecule further comprises transporting the molecule in a transport medium in a lengthwise fashion through a nanochannel to successively present each segment of a plurality of segments distributed along the length of the molecule to the NERS structure.

34. The method of claim 33, wherein transporting the molecule further comprises applying a more positive charge to the transport medium near an exit point of the nanochannel relative to a charge of the transport medium near an entrance point of the nanochannel.

35. The method of claim 30, wherein guiding at least a portion of the molecule further comprises transporting the molecule in a transport medium in a lengthwise fashion through a nanopore formed in a membrane to successively present each segment of a plurality of segments distributed along the length of the molecule to the NERS structure.

36. The method of claim 35, wherein transporting the molecule further comprises applying a more positive charge to the transport medium near an exit point of the nanopore relative to a charge of the transport medium near an entrance point of the nanopore.

37. A method of analyzing a molecule, comprising:
guiding at least a portion of the molecule in a molecule guide such that an identifiable configuration of the molecule is disposed near a molecule sensor, the molecule sensor including a NERS structure;
reacting the identifiable configuration of the molecule to a nitrogenous material disposed on the NERS structure;
irradiating the at least a portion of the molecule disposed near the molecule sensor with an excitation radiation;
detecting a Raman scattered radiation resulting from irradiation of the at least a portion of the molecule; and
detecting a nanostructure-enhanced Raman scattered radiation resulting from irradiation of the reaction between the identifiable configuration of the molecule and the nitrogenous material.

38. The method of claim 37, further comprising analyzing the Raman scattered radiation and the nanostructure-enhanced Raman scattered radiation to determine a composition of the identifiable configuration.

39. The method of claim 37, further comprising generating a wavelength of the excitation radiation that is an integer multiple of a wavelength near the nanostructure-enhanced Raman scattered radiation.

40. The method of claim 37, wherein developing the reaction comprises producing a transitory chemical bond between the nitrogenous material and the at least a portion of the molecule near the molecule sensor.

41. The method of claim 37, wherein the identifiable configuration of the molecule comprises a base selected from the group consisting of adenine, thymine, uracil, cytosine, and guanine.

42. The method of claim 37, wherein the nitrogenous material comprises a base selected from the group consisting of adenine, thymine, uracil, cytosine, and guanine.

43. The method of claim 42, wherein the nitrogenous material further comprises a material selected from the group consisting of a sugar chemically bonded to the base and a sugar-phosphate chemically bonded to the base.

44. The device of claim 37, wherein the nitrogenous material comprises an oligonucleotide and the identifiable configuration of the molecule is a complementary match to the oligonucleotide.

45. The method of claim 37, wherein guiding at least a portion of the molecule further comprises transporting the molecule in a transport medium in a lengthwise fashion through a nanochannel to successively present each segment of a plurality of segments distributed along the length of the molecule to the nitrogenous material.

46. The method of claim 45, wherein transporting the molecule further comprises applying a more positive charge to the transport medium near an exit point of the nanochannel relative to a charge of the transport medium near an entrance point of the nanochannel.

47. The method of claim 37, wherein guiding at least a portion of the molecule further comprises transporting the molecule in a transport medium in a lengthwise fashion through a nanopore formed in a membrane to successively present each segment of a plurality of segments distributed along the length of the molecule to the nitrogenous material.

48. The method of claim 47, wherein transporting the molecule further comprises applying a more positive charge to the transport medium near an exit point of the nanopore relative to a charge of the transport medium near an entrance point of the nanopore.
